(19) United States
(12) Patent Application Publication
(10) Pub. No.: US 2012/0197374 A1
(43) Pub. Date: Aug. 2, 2012

(54) Combined Stimulation with Controlled Light Distribution for Electro-Optical Cochlear Implants

(75) Inventors: Gerhard Vogt, Munchen (DE); Carolyn Garnham, Matlock Derbyshire (GB)

(73) Assignee: MED-EL ELEKTROMEDIZINISCHE GERAETE GMBH, Innsbruck (AT)

(21) Appl. No.: 13/358,599

(22) Filed: Jan. 26, 2012

Related U.S. Application Data
(60) Provisional application No. 61/436,823, filed on Jan. 27, 2011.

(51) Int. Cl. A61F 11/04; A61N 1/05

(52) U.S. Cl. 607/137; 607/116

(57) ABSTRACT

An implantable stimulation device is described which includes an implantable stimulation source carrier for insertion into or adjacent to target tissue. The stimulation source carrier includes stimulation contacts for delivering neural stimulation signals to nearby target tissue. At least one of the stimulation contacts is an optical stimulation element having multiple individual optical stimulation sub-elements for delivering optical stimulation signals to the nearby target tissue with controlled shape and direction.

This application claims priority from U.S. Provisional Patent 61/436,823, filed Jan. 27, 2011, which is incorporated herein by reference.

### TECHNICAL FIELD

The present invention relates to stimulation signals used in neural implant devices such as cochlear implants.

### BACKGROUND ART

Neural implant systems such as cochlear implants deliver stimulation signals to target neural tissue. For example, FIG. 1 shows a cochlear implant arrangement where an implant electrode 100 penetrates through a cochleostomy opening 102 into a patient cochlea 101. The intra-cochlear portion of the implant lead is referred to as the electrode array 103 and includes multiple stimulation contacts 104 that deliver electrical stimulation signals to auditory neural tissue within the cochlea 101.

Existing commercial neural implant systems are based on the use of electrical stimulation signals, but there have been some recent proposals to stimulate nerves either optically or optically in combination with electrical stimulation. The general idea is to use a light source to bring light to the nerve. A light source can be generated locally in the vicinity of the nerve (e.g. by LEDs or micro-lasers), or it can be generated remotely and transported to the nerve (e.g., by optical fiber). One challenge associated with optical stimulation is to control the shape and direction of the light field, particularly in view of the variation in rotational orientation of the light source carrier inside the cochlea.

U.S. Patent Publication 20100174329 describes a proposed arrangement for combined optical and electrical neural stimulation. The general ideas of such an arrangement are broadly discussed, but specific structural details of the optical stimulation arrangement are scant. For example, only fleeting mention is made of optical adjustment structures. Each optical stimulation contact is described as a single individual light source.

WO 2007013891 also describes an optical stimulation arrangement for cochlear implants but again seems to offer little specific discussion of controlling the optics beyond suggesting that it may be useful to arrange some combination of a mirror, lens or prism. Optical stimulation of nerves is also discussed in US 20060129210 and US 20100114190, but again, some structural details are sketchy or unaddressed.

### SUMMARY

Embodiments of the present invention are directed to an implantable stimulation device which includes an implantable stimulation source carrier for insertion into or adjacent to target tissue. The carrier includes stimulation contacts for delivering neural stimulation signals to nearby target tissue. At least one of the stimulation contacts is an electromagnetic radiation stimulation element such as an optical stimulation element having multiple individual electromagnetic radiation stimulation sub-elements for delivering electromagnetic radiation stimulation signals such as optical stimulation signals to the nearby target tissue. The electromagnetic radiation stimulation element may optionally include at least one electrical stimulation sub-element for delivering electrical stimulation signals to the nearby target tissue.

In specific embodiments, each of the electromagnetic radiation stimulation sub-elements may be individually controllable, for example, to be active or inactive. The electromagnetic radiation stimulation signals may be generated remotely from or locally at the electromagnetic radiation stimulation sub-elements. And the electromagnetic radiation stimulation sub-elements may each produce an associated electromagnetic radiation stimulation field having a given field shape such that multiple different field shapes are produced. The electromagnetic radiation stimulation element may include a shaped reflector surface for each electromagnetic radiation stimulation sub-element to differentially direct the electromagnetic radiation stimulation signals towards the nearby target tissue.

The electromagnetic radiation stimulation element may be located towards the apical end of the carrier, centrally, or towards the basal end of the carrier. The target tissue may specifically be auditory or vestibular nerve tissue or hair cells and the stimulation signals may be cochlear implant or vestibular implant stimulation signals.

Embodiments of the present invention also include a method of delivering neural stimulation signals. An implantable stimulation source carrier having multiple stimulation contacts is inserted into or adjacent to target tissue, at least one of the stimulation contacts being an electromagnetic radiation stimulation element having multiple individual electromagnetic radiation stimulation sub-elements. The stimulation contacts are then operated to deliver neural stimulation signals to nearby target tissue, including operating the electromagnetic radiation stimulation element to deliver electromagnetic radiation stimulation signals to the nearby target tissue.

Operating the electromagnetic radiation stimulation element may include individually controlling each of the electromagnetic radiation stimulation sub-elements, for example, controlling them to be active or inactive. Operating the electromagnetic radiation stimulation element may include producing for each electromagnetic radiation stimulation sub-element an associated electromagnetic radiation stimulation field having a given field shape and/or direction so that multiple, different fields are produced. Operating the stimulation contacts may also include operating at least one electrical stimulation sub-element to deliver electrical stimulation signals to the nearby target tissue.

In specific embodiments, the electromagnetic radiation stimulation signals are generated remotely from or locally at the electromagnetic radiation stimulation sub-elements. The electromagnetic radiation stimulation element or elements may include a shaped reflector surface for each electromagnetic radiation stimulation sub-element to direct the electromagnetic radiation stimulation signals towards the nearby target tissue. The electromagnetic radiation stimulation elements may be located towards an apical end of the carrier, centrally, or towards a basal end of the carrier. The target tissue may specifically be auditory nerve tissue/hair cells or vestibular nerve tissue/hair cells and the stimulation signals may be cochlear implant or vestibular implant stimulation signals.

### BRIEF DESCRIPTION OF THE DRAWINGS

FIG. 1 shows a cochlear implant stimulation arrangement.

FIG. 2 shows a side view of an implantable stimulation source carrier having optical and electrical stimulation contacts according to one or more embodiments of the present invention.

FIG. 3 A-B shows greater structural detail of an optical/electrical stimulation element according to an embodiment of the present invention.

FIG. 4 shows structural details of an alternative embodiment of an optical/electrical stimulation element.

FIG. 5 shows structural details of another alternative embodiment of an optical/electrical stimulation element.

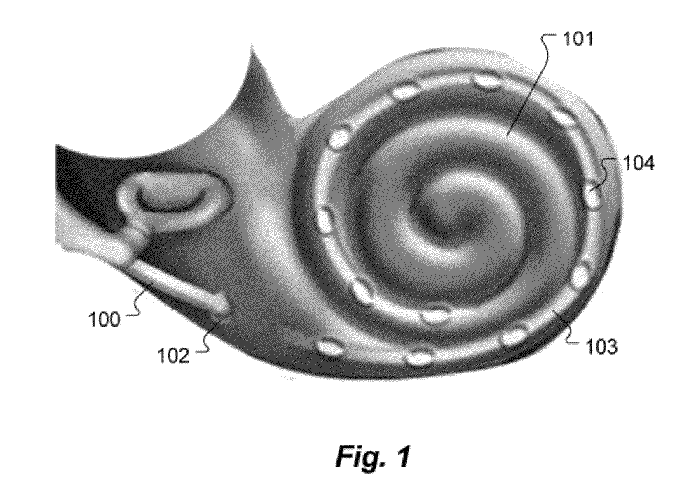

### DETAILED DESCRIPTION

Various embodiments of the present invention are directed to an implantable stimulation device which includes an implantable stimulation source carrier for insertion into or adjacent to target tissue. The stimulation source carrier includes stimulation contacts for delivering neural stimulation signals to nearby target tissue. At least one of the stimulation contacts is an electromagnetic radiation stimulation element having multiple individual electromagnetic radiation stimulation sub-elements for delivering electromagnetic stimulation signals to the nearby target tissue. The electromagnetic radiation stimulation element may optionally include at least one electrical stimulation sub-element for delivering electrical stimulation signals to the nearby target tissue. In the following description, the specific embodiments described use light signals and optical stimulation elements as the specific form of electromagnetic radiation, but it is to be understood that electromagnetic radiation broadly includes other types of electromagnetic radiation, including but not limited to infra-red radiation and ultra-violet radiation, etc.

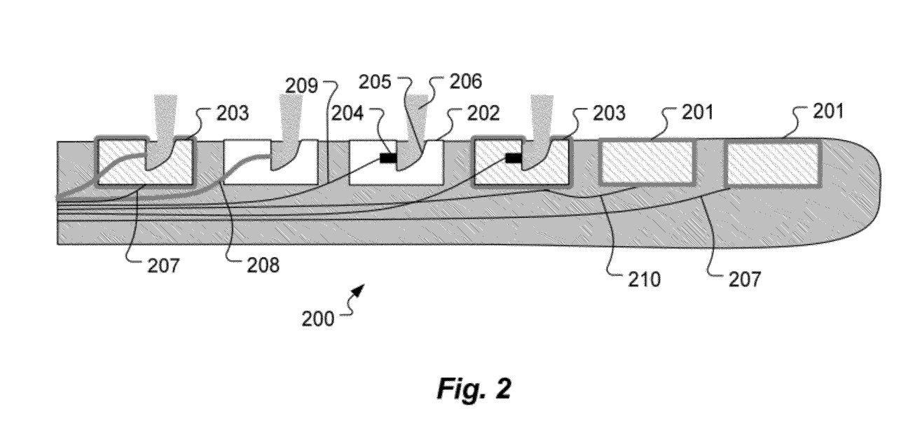

FIG. 2 shows a side view of an implantable stimulation source carrier having optical and electrical stimulation contacts according to one or more embodiments of the present invention. Stimulation source carrier 200 has three different types of stimulation elements: pure electrical by electrical stimulation contacts 201; pure optical by optical stimulation elements 202; and combined electrical and/or optical stimulation by electro-optical stimulation elements 203. The optical stimulation elements 202 and electro-optical stimulation elements 203 include multiple individual optical stimulation sub-elements that provide multiple light sources, which is not observable in the side view of FIG. 2.

The various stimulation elements—electrical stimulation contacts 201, optical stimulation elements 202, and electro-optical stimulation elements 203—are embedded into a flexible carrier material of the stimulation source carrier 200 which can be flexible and is biocompatible (e.g. medical grade silicone). In some applications, it may be useful if the carrier material is transparent in the optical wavelengths used for the optical stimulation. The stimulation elements can be arranged in the stimulation source carrier 200 in various geometric distributions according to the needs of the given application. For example, improved speech understanding based on a hybrid electro-optical speech encoding strategy may benefit from a higher density of optical stimulation elements 202 towards the apical end of the stimulation source carrier 200. However, for other stimulation strategies, it might be more useful the other way round, with a higher density of optical stimulation elements 202 towards the basal or central end of the stimulation source carrier 200.

The electrical stimulation contacts 201, optical stimulation elements 202, and electro-optical stimulation elements 203 can also be arranged in various three-dimensional geometries within the carrier material of the stimulation source carrier 200. In many cases it is advantageous if the surfaces of the electrical stimulation contacts 201 are in contact with the surrounding fluids or tissue. And due to the higher spread of the electrical field, the electrical stimulation contacts 201 can also be placed on the opposite side (lateral wall orientation) of the optical stimulation elements 202 and/or electro-optical stimulation elements 203. But in any case the placement of the electrical stimulation contacts 201 should not hinder the light generated by the optical stimulation elements 202 or electro-optical stimulation elements 203.

The electrical stimulation contacts 201 can be made from standard materials used for this purpose, e.g. platinum. Each electrical stimulation contact 201 is connected with a current or voltage source by a connecting wire 207 of a biocompatible conductive material such as platinum-iridium alloy. The connecting wires 207 typically are coated with an insulator (e.g., polytetrafluoroethylene (PTFE)), although alternatively the material of the stimulation source carrier 200 may have suitable electrical insulation properties so long as the connecting wires 207 do not make direct physical contact. The connecting wires 207 transport the electrical charge that is finally responsible for the creation of a suitable electrical field using a second ground electrode (not shown in FIG. 2).

The electrical stimulation contacts 201 can also be part of an integrated circuit that might control various elements. The electrical stimulation contacts 201 can have various specific shapes to best suit the particular application. For example, cochlear implant electrode contacts typically are a circle shape that is bent to follow the cylindrical shape of the stimulation source carrier 200. It might be useful to have some other structured electrode contact geometry to realize a greater surface area and thereby provide better electrical stimulation. The size and shape of the electrical stimulation contacts 201 do not necessarily need to be similar to the electro-optical stimulation elements 203.

The optical stimulation elements 202 and the electro-optical stimulation elements 203 may contain multiple local light sources 204 that emit electromagnetic radiation stimulation signals. The local light sources 204 can be various types of light emitting diodes (LEDs), micro-LEDs, vertical-cavity surface-emitting lasers (VCSELs), laser diodes (LDs), lasers or other devices that emit optical radiation. The optical stimulation signal may be delivered to the local light sources 204 by connecting wire 209. Specific local light sources 204 may have an intrinsic collimation by being placed together with a shaped reflector surface 205, which may be created, for example, on the side walls and/or the bottom of the optical stimulation elements 202 and the electro-optical stimulation elements 203.

Rather than local light sources 204, the optical stimulation elements 202 and the electro-optical stimulation elements 203 may deliver remotely generated optical stimulation signals that are delivered to them by optical fibers 208. Optical fibers 208 of different specific materials are advantageous for different specific applications such as light guiding in specific spectral regions, high flexibility, low transmission losses, etc. Such optical fibers 208 can be made of SiO₂ or other glass-type materials or of various polymers such as polymer optical fibers (POF). There are also a wide variety of different types of fiber that can be used for light guiding such as fiber bundles, hollow fibers, photonic crystal fibers, multi-core fibers and similar variants.

The shaped reflector surface 205 directs and shapes the light of the optical stimulation signal to form the desired optical stimulation field 206. The shaped reflector surfaces 205 can be formed in the body of the optical stimulation elements 202 and the electro-optical stimulation elements 203, for example, by injection molding of a suitable polymer and subsequent coating with a suitable reflective material. A mold can also directly be filled with a suitable metal that reflects the light. Or the structure of the shaped reflector surface 205 can alternatively be achieved by stamping it into a suitable metal sheet or by closed-die coining or other similar technologies.

If the surface of the shaped reflector surface 205 is covered by or made of metal, then it may also be suitable for delivering electrical stimulation signals by attaching a connecting wire 207 to it. In any event, the electro-optical stimulation elements 203 receive a connecting wire 207 for connecting the electric stimulation signals to the electric stimulation functionality. Multiple elements for electrical stimulation can be connected with a twisted wire 210 since some minimum size electrical contact surface is required to generate a suitable electrical field for reasons of charge density limitations or field dimension. The individual wires 207, 209 and 210 and/or optical fibers 208 can be grouped into mixed bundles of fibers and wires or bundles of fibers and bundles of wires.

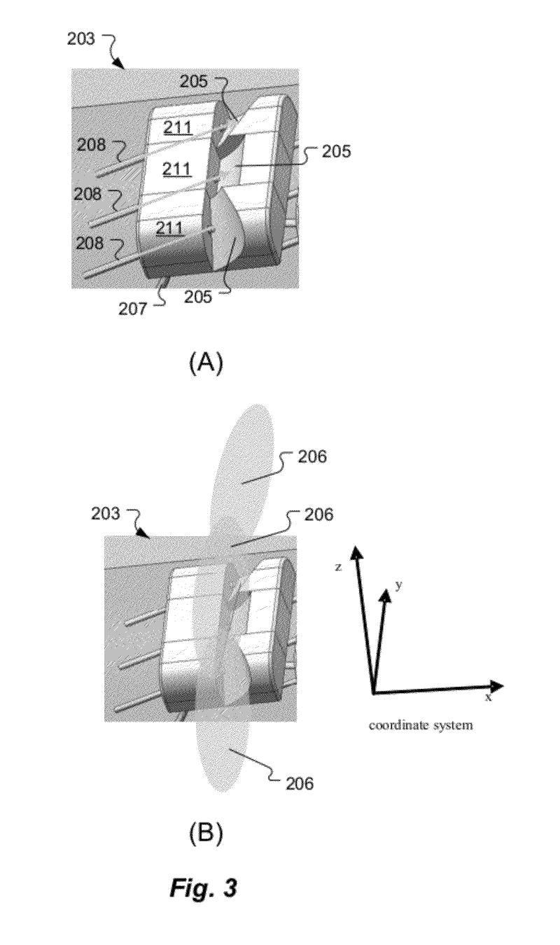

FIG. 3 A-B shows greater structural detail of an electro-optical stimulation element 203 according to an embodiment of the present invention, details of which may also be present in the optical stimulation elements 202. The electro-optical stimulation element 203 is subdivided into three separate individual optical stimulation sub-elements 211 which each contain a corresponding light source, here shown as optical fibers 208. Of course, other specific embodiments may have different numbers of individual optical stimulation sub-elements 211. Light exiting the optical fibers 208 falls onto individual shaped reflector surfaces 205 that produce corresponding individual optical stimulation fields 206. The light sources such as the optical fibers 208 do not need to lie specifically as shown in FIG. 3 within a flat plane, but for example, can also be arranged on the surface of a cylinder. The individual shaped reflector surfaces 205 are arranged according the orientation of the optical fibers 208.

The end surfaces of the optical fibers 208 can either be flat or terminate in micro-lenses. Optical fibers 208 with curved refractive end surfaces or micro-lenses on top of the fiber exit are termed “lensed fibers”. Flat fiber exit faces can have any angle. Optical fibers 208 can be inserted through holes within the electro-optical stimulation element 203. If the end face of the optical fiber 208 is rotationally symmetric (e.g. a lens or a flat right angle surface), then alignment of the fiber is only necessary in one dimension along the fiber's optical axis. Fixation of the optical fibers 208 can be achieved, for example, using the carrier material (e.g. optical and medical-grade silicone) of the stimulation source carrier 200, or medical grade epoxy.

If the carrier material of the stimulation source carrier is not transparent in the required optical wavelength, the gaps in front of the shaped reflector surfaces 205 can be left open, or be filled with a suitable transparent and biocompatible material such as an optical grade biocompatible silicone or epoxy. Because such fillings are restricted to the small compartment in front of the shaped reflector surfaces 205, the flexibility of the stimulation source carrier 200 is not affected. Standard procedures similar to those used in the assembly of conventional cochlear implant electrodes can be used to keep the optical surfaces free of the carrier material when the stimulation source carrier 200 is produced.

If the electro-optical stimulation element 203 is made of or coated with an electrically conductive material, then it can also be used for delivering electrical stimulation signals. The electro-optical stimulation element 203 can also have multiple different surface coatings; for example, a polymer base material might be covered with an electrically conductive material (e.g., Platinum), which itself is coated at the shaped reflector surfaces 205 with another coating that has a high reflectivity in the desired wavelength regime, such as gold or silver. By connecting a conductive part of the electro-optical stimulation element 203 with a suitable connecting wire 207, the necessary electric charge can be loaded onto the element. Of course, the electro-optical stimulation element 203 does not need to be used for electrical stimulation, and thus could function as a pure optical stimulation element 202. In that case, electrically conductive coatings and connecting wires 207 are not needed.

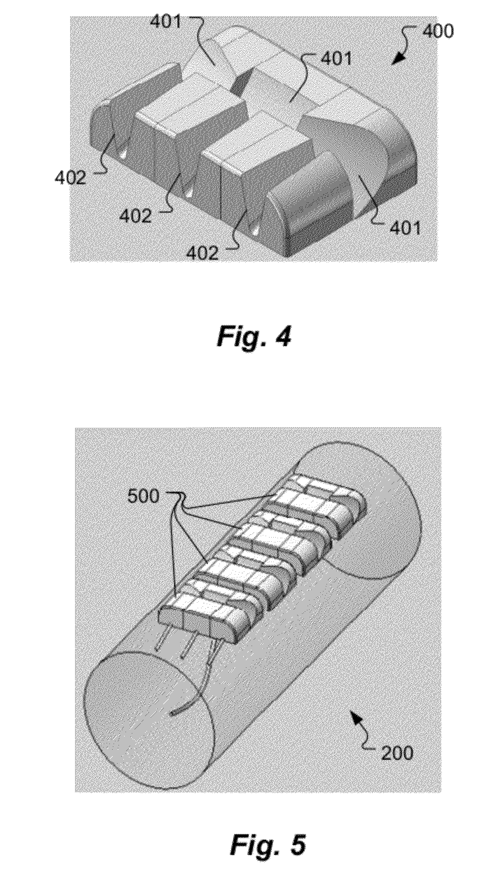

FIG. 4 shows structural details of an alternative embodiment of an optical stimulation element 400 where the optical fibers 208 are held by v-shaped fiber grooves 401. These facilitate the insertion of the optical fibers 208, which can be easily dropped into the v-shaped fiber grooves 401 and then fixed into place (e.g. using silicone or epoxy as discussed above). If the optical fibers 208 are rotationally invariant along their optical axis, then no rotational alignment is needed after positioning.

FIG. 4 also shows that the geometry of the shaped reflector surfaces 401 can be varied to enable a high variety of differently shaped light fields 206. For example, the light field 206 can be collimated along one or two axes, and/or focused or directed in different directions. Of course, additional optical elements also can be employed along the light path such as lenses, gratings, zone plates and the like. Specific embodiments may also use optical fibers 208 whose outcouple surfaces are angled to guide the light to the side (“side-firing fibers”), either by total reflection or by additional mirror coatings on the front faces of the optical fibers 208. Side-firing fibers also include optical fibers 208 with optics at their tip such as lenses, prisms and mirrors. In some such embodiments, the shaped reflector surfaces 401 may not be of direct optical use, but they may still help greatly during alignment. Side-firing optical fibers 208 can be placed into the v-shaped fiber grooves 402, or into fiber openings, and they will need to be rotationally aligned with regards to the direction of the principal ray. Alignment may also be facilitated by suitable shaped reflector surfaces 401 that pre-orient the optical fibers 208.

After insertion of the stimulation source carrier 200 into or adjacent to the target nerve tissue (e.g., the scala tympani or scala vestibuli of the cochlea (or the semicircular canals or otolith organs of the vestibular system), it is aligned with its rotational axis parallel to the central axis of the scala. For optical stimulation, the light has to be precisely directed onto the correct region of the nerves or hair cells themselves. Correct alignment can be easily verified by standard methods of measuring the optically evoked nerve responses with the electrical stimulation contacts 201 or the electrical contact surfaces of the electro-optical stimulation element 203.

A rough orientation of the stimulation source carrier 200 can be achieved during the surgical insertion procedure (e.g. using a placement holding silicone ‘wing’). but the stimulation source carrier 200 could still twist out of its optimal position. With a stimulation source carrier 200 of isotropic flexibility, such a twist can be maintained in the order of few tens of degrees using standard technology. Isotropicity is meant here in terms of equal flexibility in either direction perpendicular to the rotational axis. Such isotropicity can easily be broken by adding elements that have a different flexibility in various directions. By this methodology, twisting of the stimulation source carrier 200 can be further reduced.

Even with proper insertion orientation and a low twist of the stimulation source carrier 200, an optimal alignment of a one-dimensional light source array is unlikely. The stimulation source carrier 200 might have varying distances to the target tissue, the nerve itself might not follow a perfect helical geometry or have regions of nonfunctional tissue, and other (as yet unknown) effects will require control of the light field. The required control of the light field distribution can be achieved by employing a multi-dimensional array of specifically adapted light fields as with the electro-optical stimulation elements 203 and optical stimulation elements 202 described herein. After insertion of the stimulation source carrier 200, only the particular light sources whose light fields best cover the target nerve tissue are used. This saves energy and unnecessary illumination of non-nerve tissue is greatly reduced.

It might be advantageous for the light fields 206 of the different individual light sources to have different aspect ratios along the x- and y-axes as shown in FIG. 3B. This provides high spatial selectivity in the x-direction while at the same time adequately illuminating the nerve. Ideally only one of the multiple individual light sources may be needed, and even in the worst case, two equally split light sources will suffice (requiring parallel simultaneous stimulation using these two channels at a controlled intensity ratio).

FIG. 5 shows structural details of another alternative embodiment of another optical stimulation element 500 where four electro-optical stimulation elements are linearly combined together to form a single larger optical stimulation element 500. The optical stimulation element 500 might also be electrically connected internally to form a single larger electronic contact. A larger optical stimulation element 500 with many individual light sources and reflectors in both the x- and the y-direction allows effective optical targeting of multiple different nerve tissue regions. Each line of light sources and reflectors in the y-direction can be used to illuminate a different spot on the target tissue. Because of the possible high spatial resolution for optical stimulation and the generally lower spatial resolution for electrical stimulation, multiple individually addressable optical stimulation elements may be combinable with fewer electrical stimulation elements.

One advantage of a larger optical stimulation element 500 having multiple lines of light sources in the x-direction would be a simpler handling during manufacturing of the stimulation source carrier 200. Such an optical stimulation element 500 might usefully have a curved geometry in one direction, for example, to follow the natural geometry of the cochlea, or be flexible. The size of the optical stimulation element 500 however will have some upper limit to avoid overly limiting the flexibility of the whole stimulation source carrier 200.

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable stimulation device comprising:
an implantable stimulation source carrier for insertion into or adjacent to target tissue, the stimulation source carrier having a plurality of stimulation contacts for delivering neural stimulation signals to nearby target tissue;
wherein at least one of the stimulation contacts comprises an electromagnetic radiation stimulation element having a plurality of individual electromagnetic radiation stimulation sub-elements for delivering electromagnetic radiation stimulation signals to the nearby target tissue.

2. A stimulation device according to claim 1, wherein each of the electromagnetic radiation stimulation sub-elements is individually controllable.

3. A stimulation device according to claim 2, wherein at least one of the electromagnetic radiation stimulation sub-elements is controllable to be inactive.

4. A stimulation device according to claim 1, wherein the electromagnetic radiation stimulation sub-elements each produce an associated electromagnetic radiation stimulation field having a given field shape or direction, and wherein a plurality of different field shapes and directions are produced.

5. A stimulation device according to claim 1, wherein the electromagnetic radiation stimulation element further comprises at least one electrical stimulation sub-element for delivering electrical stimulation signals to the nearby target tissue.

6. A stimulation device according to claim 1, wherein the electromagnetic radiation for electromagnetic radiation stimulation is generated remotely from the electromagnetic radiation stimulation sub-elements.

7. A stimulation device according to claim 1, wherein the electromagnetic radiation for electromagnetic radiation stimulation is generated locally at the electromagnetic radiation stimulation sub-elements.

8. A stimulation device according to claim 1, wherein the electromagnetic radiation stimulation element includes a shaped reflector surface for each electromagnetic radiation stimulation sub-element to differentially direct the electromagnetic radiation stimulation signals towards the nearby target tissue.

9. A stimulation device according to claim 1, wherein the electromagnetic radiation stimulation element is located towards an apical end of the stimulation source carrier.

10. A stimulation device according to claim 1, wherein the electromagnetic radiation stimulation element is located towards a basal end of the stimulation source carrier.

11. A stimulation device according to claim 1, wherein the target tissue is auditory nerve tissue or hair cells and the stimulation signals are cochlear implant stimulation signals.

12. A stimulation device according to claim 1, wherein the target tissue is vestibular nerve tissue or hair cells and the stimulation signals are vestibular implant stimulation signals.

13. A stimulation device according to claim 1, wherein the electromagnetic stimulation signals include optical stimulation signals.

14. A method of delivering neural stimulation signals comprising:
inserting into or adjacent to target tissue an implantable stimulation source carrier having a plurality of stimulation contacts, wherein at least one of the stimulation contacts comprises an electromagnetic radiation stimulation element having a plurality of individual electromagnetic radiation stimulation sub-elements; and
operating the stimulation contacts to deliver neural stimulation signals to nearby target tissue, including operating the electromagnetic radiation stimulation element to deliver electromagnetic radiation stimulation signals to the nearby target tissue.

15. A method according to claim 14, wherein operating the electromagnetic radiation stimulation element includes individually controlling each of the electromagnetic radiation stimulation sub-elements.

16. A method according to claim 15, wherein at least one of the electromagnetic radiation stimulation sub-elements is controlled to be inactive or the intensity is varied between the light sources of a sub-array.

17. A method according to claim 14, wherein operating the electromagnetic radiation stimulation element includes producing for each electromagnetic radiation stimulation sub-element an associated electromagnetic radiation stimulation field having a given field shape or direction, and wherein a plurality of different field shapes or directions are produced.

18. A method according to claim 14, wherein operating the stimulation contacts includes operating at least one electrical stimulation element to deliver electrical stimulation signals to the nearby target tissue.

19. A method according to claim 14, wherein the electromagnetic radiation for electromagnetic radiation stimulation is generated remotely from the electromagnetic radiation stimulation sub-elements.

20. A method according to claim 14, wherein the electromagnetic radiation for electromagnetic radiation stimulation is generated locally at the electromagnetic radiation stimulation sub-elements.

21. A method according to claim 14, wherein the electromagnetic radiation stimulation element includes a shaped reflector surface for each electromagnetic radiation stimulation sub-element to direct the electromagnetic radiation stimulation signals towards the nearby target tissue.

22. A method according to claim 14, wherein the electromagnetic radiation stimulation element is located towards an apical end of the stimulation source carrier.

23. A method according to claim 14, wherein the electromagnetic radiation stimulation element is located towards a basal end of the stimulation source carrier.

24. A method according to claim 14, wherein the target tissue is auditory nerve tissue or hair cells and the stimulation signals are cochlear implant stimulation signals.

25. A method according to claim 14, wherein the target tissue is vestibular nerve tissue or hair cells and the stimulation signals are vestibular implant stimulation signals.

26. A method according to claim 14, wherein the electromagnetic stimulation signals include optical stimulation signals.
